(19) United States
(12) Patent Application Publication
(10) Pub. No.: US 2022/0110865 A1
(43) Pub. Date: Apr. 14, 2022

(54) METHOD FOR MANUFACTURING ORALLY DISINTEGRATING TABLET, AND ORALLY DISINTEGRATING TABLET

(71) Applicant: SAWAI PHARMACEUTICAL Co., Ltd., Osaka (JP)

(72) Inventors: Kohei IKUNO, Osaka City (JP); Sae UCHIDA, Osaka City (JP)

(21) Appl. No.: 17/560,819

(22) Filed: Dec. 23, 2021

Related U.S. Application Data
(63) Continuation of application No. PCT/JP2020/025940, filed on Jul. 2, 2020.

(30) Foreign Application Priority Data
Jul. 4, 2019 (JP) 2019-125352

(51) Int. Cl. A61K 9/00; A61K 9/20

(52) U.S. Cl. CPC A61K 9/0056; A61K 9/2027; A61K 9/205; A61K 9/2054

(57) ABSTRACT

A method for manufacturing an orally disintegrating tablet, in which adequate hardness for preventing breakage of the tablet during transport or handling by a medical practitioner or a patient is provided while rapid disintegration is maintained. The method for manufacturing an orally disintegrating tablet according to an embodiment of the present invention comprises spraying or dropping a liquid including hydroxypropyl cellulose having a viscosity in a 2% aqueous solution at 20° C. of 150 mPa·s or more and 400 mPa·s on additives, and performing fluid bed granulation. The liquid including hydroxypropyl cellulose may be sprayed or dropped on the additives so that the content of the hydroxypropyl cellulose with respect to 100 wt % of the orally disintegrating tablet is 0.2 wt % to 5 wt %.

### CROSS-REFERENCE TO RELATED APPLICATIONS

This application is a Continuation of International Patent Application No. PCT/JP2020/025940, filed on Jul. 2, 2020, which claims priority to Japanese Patent Application No. 2019-125352, filed on Jul. 4, 2019, the disclosures of which are incorporated herein by reference for all purposes as if fully set forth herein.

### FIELD

The present invention relates to a method for manufacturing an orally disintegrating tablet, and an orally disintegrating tablet by the method. In particular, the present invention relates to a method for manufacturing an orally disintegrating tablet which has both rapid disintegration and adequate hardness.

### BACKGROUND

An orally disintegrating tablet is a tablet that easily disintegrates in the oral cavity and is a useful preparation particularly for the elderly and young children who have poor swallowing ability. The orally disintegrating tablet is required to disintegrate rapidly in the oral cavity, and it is desirable to disintegrate within 30 seconds in the oral cavity, for example. On the other hand, in addition to the orally disintegrating tablet, solid tablets are generally required to have a certain degree of hardness to prevent breakage of the tablet during transport or handling by a medical practitioner or a patient. Therefore, the development of the orally disintegrating tablet has a major problem of providing the orally disintegrating tablet with rapid disintegration and high tablet hardness, which are contradictory properties.

For example, Japanese Patent No. 3274416 describes an orally disintegrating granular preparation containing (1) an active ingredient, (2) a sugar alcohol selected from a group consisting of erythritol, xylitol, and sorbitol, and (3) a polyvinylpyrrolidone having a number average molecular weight of 20,000 to 50,000 and having a water activity value of 15 or less.

### SUMMARY

A method for manufacturing an orally disintegrating tablet having adequate hardness for preventing breakage of the tablet during transport or handling by a medical practitioner or a patient is provided while rapid disintegration is maintained.

According to an embodiment of the present invention, a method for manufacturing an orally disintegrating tablet is provided including performing fluid bed granulation while splaying or dropping a liquid including hydroxypropyl cellulose having a viscosity in a 2% aqueous solution at 20° C. of 150 mPa·s or more and 400 mPa·s or less on additives.

The liquid including the hydroxypropyl cellulose may be sprayed or dropped on the additives so that a content of the hydroxypropyl cellulose with respect to 100 wt % of the orally disintegrating tablet is 0.2 wt % or more and 5 wt % or less.

The liquid including the hydroxypropyl cellulose may be sprayed or dropped on the additives so that a content of the hydroxypropyl cellulose with respect to 100 wt % of the orally disintegrating tablet is 0.2 wt % or more and 2 wt % or less.

The liquid including the hydroxypropyl cellulose is prepared by dissolving the hydroxypropyl cellulose and a sweetener, and the additives may be prepared by mixing an excipient and a disintegrant.

A pharmaceutically active ingredient may be dissolved or dispersed in the liquid including the hydroxypropyl cellulose, or the pharmaceutically active ingredient may be mixed with the additives.

According to an embodiment of the present invention, there is provided an orally disintegrating tablet is provided including a particle including hydroxypropyl cellulose having a viscosity in a 2% aqueous solution at 20° C. of 150 mPa·s or more and 400 mPa·s or less, wherein a bulk density of the particles is 0.40 g/cm³ or less.

A content of the hydroxypropyl cellulose with respect to 100 wt % of the orally disintegrating tablet may be 0.2 wt % or more and 5 wt % or less.

A content of the hydroxypropyl cellulose with respect to 100 wt % of the orally disintegrating tablet may be 0.2 wt % or more and 2 wt % or less.

### BRIEF DESCRIPTION OF DRAWINGS

FIG. 1 is a flow diagram illustrating a method for manufacturing an orally disintegrating tablet according to an embodiment.

### DESCRIPTION OF EMBODIMENT

Hereinafter, a method for manufacturing an orally disintegrating tablet according to the present invention and an orally disintegrating tablet will be described. However, the method for manufacturing the orally disintegrating tablet of the present invention and the orally disintegrating tablet are not to be construed as being limited to the contents of the embodiments and examples described below.

As a result of studies by the present inventors, it has been found that an orally disintegrating tablet having adequate hardness for preventing breakage of the tablet during transport or handling by a medical practitioner or a patient while rapid disintegration is maintained can be manufactured by performing fluid bed granulation using a granulation liquid including a hydroxypropyl cellulose of a specific grade.

### [Method for Manufacturing Orally Disintegrating Tablet]

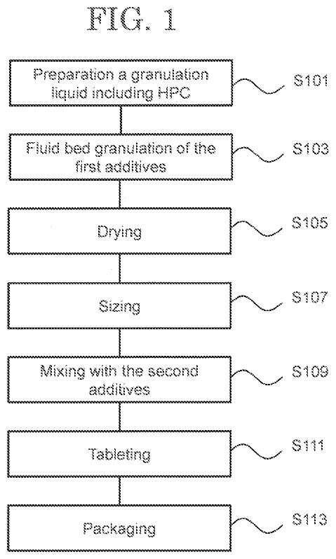

The orally disintegrating tablet according to the present invention can be manufactured by performing fluid bed granulation while spraying or dropping a liquid including a hydroxypropyl cellulose (hereinafter, also referred to as HPC) having a viscosity in a 2% aqueous solution at 20° C. of 150 mPa·s or more and 400 mPa·s or less on additives. FIG. 1 is a flow diagram illustrating a method for manufacturing an orally disintegrating tablet according to an embodiment of the present invention.

When using HPC as a binder in manufacturing an orally disintegrating tablet, it was common to use a low-viscosity grade HPC to prevent deterioration of disintegration. For example, SSL (viscosity in a 2% aqueous solution at 20° C. is 2 mPa·s or more and 2.9 mPa·s or less, average molecular weight 40,000), SL (viscosity in a 2% aqueous solution at 20° C. is 3 mPa·s or more and 5.9 mPa·s or less, average molecular weight 100,000), and L (viscosity in a 2% aqueous solution at 20° C. is 6 mPa·s or more and 10 mPa·s or less, average molecular weight 140,000) of Nippon Soda Co., Ltd. are commercially available as the low-viscosity grade HPC. However, even if these low-viscosity grade HPCs are used, it is difficult to obtain adequate hardness for preventing the breakage of the tablet during transport or handling by a medical practitioner or a patient. For example, H (viscosity in a 2% aqueous solution at 20° C. is 1,000 mPa·s or more and 4,000 mPa·s or less, average molecular weight 1,000,000), VH (viscosity in a 2% aqueous solution at 20° C. is 4,001 mPa·s or more and 6,000 mPa·s or less, average molecular weight 2,500,000) of Nippon Soda Co., Ltd. are commercially available as a high-viscosity grade HPC in which the viscosity in a 2% aqueous solution at 20° C. exceeds 400 mPa·s. However, these high-viscosity grade HPCs are not preferable because they have high viscosity when the granulation liquid is prepared and are inferior in manufacturability. The average molecular weight shall be measured by the gel permeation chromatography (GPC) method.

For example, M of Nippon Soda Co., Ltd can be used as the HPC having a viscosity in a 2% aqueous solution at 20° C. of 150 mPa·s or more and 400 mPa·s or less. The average molecular weight is 700,000.

In an embodiment, a pharmaceutically acceptable known solvent capable of dissolving HPC is used as a solvent used for preparing a liquid including the HPC having a viscosity in a 2% aqueous solution at 20° C. of 150 mPa·s or more and 400 mPa·s or less. For example, purified water, an organic solvent such as methanol, ethanol, isopropanol, propylene glycol, or methylene chloride, and a mixed solution of purified water and these organic solvents can be used as the solvent, but not limited thereto.

In an embodiment, a liquid including the HPC having the viscosity in a 2% aqueous solution at 20° C. of 150 mPa·s or more and 400 mPa·s or less is sprayed or dropped on the additives so that the content of HPC with respect to 100 wt % of the orally disintegrating tablet is 0.2 wt % or more and 5 wt % or less. In an embodiment, the liquid including the HPC may be sprayed or dropped on the additives so that the content of HPC with respect to 100 wt % of the orally disintegrating tablet is 0.2 wt % or more and 2 wt % or less.

In an embodiment, the liquid including the HPC may include a sweetener. A pharmaceutically acceptable known additive which can be dissolved in the above solvents can be used as a sweetener. Aspartame, sweet hydrangea leaf, powdered sweet hydrangea leaf, liquid sugar, fructose, fructose glucose syrup, reduced maltose syrup, glycyrrhiza, glycyrrhiza extract, crude glycyrrhiza extract, powdered glycyrrhiza, xylitol, glycine, glycerin, dipotassium glycyrrhizinate, disodium glycyrrhizinate, monoammonium glycyrrhizinate, brown sugar, high fructose syrup, high dextrose starch syrup, saccharin, sodium saccharin hydrate, sucralose, stevia extract, stevia extracted purified product, purified sucrose, purified sucrose spheres, purified honey, D-sorbitol, D-sorbitol solution, simple syrup, lactose hydride, concentrated glycerin, white sugar, honey, glucose, high-fructose corn syrup, powder reducing maltose syrup, maltitol, maltitol solution, maltose hydride, D-mannitol, and starch syrup are exemplified as the sweetener, but not limited thereto. Although there is no particular limitation on the content of the sweetener, a trace amount capable of masking the bitterness or the like of the other additive may be added.

The HPC having a viscosity in a 2% aqueous solution at 20° C. of 150 mPa·s or more and 400 mPa·s or less and a sweetener are dissolved in a solvent to prepare a granulation liquid including the HPC (S101).

For example, an excipient and a disintegrant are used as additives (first additives) used for the fluid bed granulation. A known additive added to the orally disintegrating tablet is used as the excipient and the disintegrant. Lactose, crystalline cellulose, D-mannitol, erythritol, xylitol, sorbitol, isomalt, maltitol, white sugar, sucrose, glucose, and partially pregelatinized starch are exemplified as the excipient, but not limited thereto. One or more of these additives can be used as the excipient. The excipient is preferably contained in an amount of 50.0 wt % or more and 97.0 wt % or less with respect to 100 wt % of the orally disintegrating tablet.

Low-substituted hydroxypropyl cellulose, carmellose, calcium carmellose, sodium carmellose, hydroxypropylstarch, sodium carboxymethyl starch, crospovidone, powdered agar, and partially pregelatinized starch are exemplified as the disintegrant, but not limited thereto. One or more of these additives can be used as the disintegrant.

The excipient and the disintegrant are supplied to a fluid bed granulator, and the granulation liquid is sprayed or dropped to be subjected to fluid bed granulation (S103). The granulated substance is dried (S105).

In an embodiment, a pharmaceutically active ingredient may be dissolved or dispersed in the liquid including the HPC. A pharmaceutically active ingredient may be mixed with the additives, which are subjected to fluid bed granulation. The pharmaceutically active ingredient contained in the orally disintegrating tablet is not particularly limited, and an orally disintegrating tablet can be manufactured using a known ingredient.

The dried granulated substance is sieved to regulate the particle size (S107). The sized powder is mixed with other additives (second additives) to obtain a pre-tableting powder (S109).

Disintegrants, fluidizers, and lubricants are used as the other additive. One or more of the disintegrants described above can be selected as the disintegrant. The disintegrant is preferably contained in an amount of 1.0 wt % or more and 40.0 wt % or less with respect to 100 wt % of the orally disintegrating tablet in the sum of adding to the first additives and the second additives.

A known additive added to an orally disintegrating tablet can be used as the fluidizer and lubricant. Magnesium aluminometasilicate, hydrated silicon dioxide, light anhydrous silicic acid, and calcium silicate are exemplified as the fluidizer, but not limited thereto. One or more of these additives can be used as the fluidizer. The fluidizer is preferably contained in an amount of 0.1 wt % or more and 5.0 wt % or less with respect to 100 wt % of the orally disintegrating tablet. Further, light silicic acid anhydride, magnesium stearate, calcium stearate, stearyl fumarate sodium, talc, hydrogenated oil, and the like are exemplified as the lubricant, but not limited thereto. One or more of these additives can be used as the lubricant. The lubricant is preferably contained in an amount of 0.1 wt % or more and 5.0 wt % or less with respect to 100 wt % of the orally disintegrating tablet.

The pre-tableting powder is supplied to a tableting machine and tableted (S111). In this way, an orally disintegrating tablet can be manufactured. An appropriate packaging is provided to the prepared orally disintegrating tablet (S113).

### [Orally Disintegrating Tablet]

The orally disintegrating tablet manufactured by the manufacturing method according to the present invention contains particles containing the HPC having a viscosity in a 2% aqueous solution at 20° C. of 150 mPa·s or more and 400 mPa·s or less, and the bulk density of the particle is 0.40 g/cm³ or less. The particles are particles constituting a size regulated powder. Since the size regulated powder contains the HPC having such viscosity and has such a small bulk density, it is possible to have adequate hardness for preventing breakage of the tablet during transport or handling by a medical practitioner or a patient while rapid disintegration is maintained.

### EXAMPLES

### Example 1

As Example 1, an orally disintegrating tablet in which the content of HPC with respect to 100 wt % of the orally disintegrating tablet is 0.02 wt % is manufactured. 0.06 g of hydroxypropyl cellulose (HPC-M, Nippon Soda Co., Ltd.) and 3 g of sucralose (San-Ei Gen F.F.I., Inc.) were dissolved in purified water to prepare 276 g of a granulation liquid. 243 g of D-mannitol (Mannite P, Mitsubishi Shoji Food Tech Co., Ltd.), 18 g of low-substituted hydroxypropyl cellulose (L-HPC (NBD-22), Shin-Etsu Chemical Co., Ltd.) and 18 g of carmellose (NS-300®, Nichirin Chemical Industries, Ltd.) were supplied to a fluid bed granulator (manufactured by Powrex Corporation, model: MP-01) and mixed. Fluid bed granulation was performed by spraying the granulation liquid on the mixed powder. The granulated substances were dried at 70° C. The size of the dried granulated substances was regulated by a sieve No. 22 to obtain a sized regulated powder. The sized powder, 6 g of crospovidone (Kollidon® CL-F, BASF), 9 g of magnesium aluminometasilicate (Neusilin® UFL2, Fuji Chemical Industries Co., Ltd.), and 3 g of magnesium stearate (Taihei Chemical Industrial Co., Ltd.) were mixed to obtain a pre-tableting powder. Using a tableting machine (VELA5, manufactured by Kikusui Seisakusho Ltd.), to obtain an orally disintegrating tablet of Example 1 having a thickness of 2.10 mm.

### Example 2

As Example 2, the additive amount of HPC-M was changed to 0.6 g, and the additive amount of D-mannitol was changed to 242.4 g to manufacture an orally disintegrating tablet in which the content of HPC-M is 0.2 wt %.

### Example 3

As Example 3, the additive amount of HPC-M was changed to 1.5 g, and the additive amount of D-mannitol was changed to 241.5 g to manufacture an orally disintegrating tablet in which the content of HPC-M is 0.5 wt %.

### Example 4

As Example 4, the additive amount of HPC-M was changed to 3.0 g, and the additive amount of D-mannitol was changed to 240.0 g to manufacture an orally disintegrating tablet in which the content of HPC-M is 1.0 wt %.

### Example 5

As Example 5, the additive amount of HPC-M was changed to 6.0 g, the additive amount of purified water was increased to prepare 402 g of a granulation liquid, and the additive amount of D-mannitol was changed to 237.0 g to manufacture an orally disintegrating tablet in which the content of HPC-M is 2.0 wt %.

### Comparative Example 1

As Comparative Example 1, the grade of HPC was changed to HPC-SSL to manufacture an orally disintegrating tablet in which the content of HPC is 0.02 wt %. An orally disintegrating tablet of Comparative Example 1 having a thickness of 2.10 mm was obtained by the same manufacturing method as in Example 1, except that 0.06 g of HPC-SSL (Nippon Soda Co., Ltd.) was used instead of HPC-M.

### Comparative Example 2

As Comparative Example 2, the additive amount of HPC-SSL was changed to 0.6 g, and the additive amount of D-mannitol was changed to 242.4 g to manufacture an orally disintegrating tablet in which the content of HPC-SSL is 0.2 wt %.

### Comparative Example 3

As Comparative Example 3, the additive amount of HPC-SSL was changed to 3.0 g, and the additive amount of D-mannitol added was changed to 240.0 g to manufacture an orally disintegrating tablet in which the content of HPC-SSL is 1.0 wt %.

### Comparative Example 4

As Comparative Example 4, the additive amount of HPC-SSL was changed to 6.0 g, and the additive amount of D-mannitol was changed to 237.0 g to manufacture an orally disintegrating tablet in which the content of HPC-SSL is 2.0 wt %.

### Comparative Example 5

As Comparative Example 5, the grade of HPC was changed to HPC-L to manufacture an orally disintegrating tablet in which the content of HPC is 0.5 wt %. The orally disintegrating tablet of Comparative Example 5 having a thickness of 2.10 mm was obtained by the same manufacturing method as in Example 3, except that 1.5 g of HPC-L (Nippon Soda Co., Ltd.) was used instead of HPC-M.

### Comparative Example 6

As Comparative Example 6, an orally disintegrating tablet in which the content of HPC-L is 1.0 wt % was manufactured by the same manufacturing method as in Example 4, except that HPC-L was used.

### Comparative Example 7

As Comparative Example 7, the grade of HPC was changed to HPC-H to manufacture an orally disintegrating tablet in which the content of HPC is 1.0 wt %. The orally disintegrating tablet of Comparative Example 7 having a thickness of 2.10 mm was obtained by the same manufacturing method as in Example 4, except that 3.0 g of HPC-H (Nippon Soda Co., Ltd.) was used instead of HPC-M.

### [Hardness]

The hardness of each of the three tablets was measured for the orally disintegrating tablets of Examples 1 to 5 and Comparative Examples 1 to 7 by a Schleuniger tablet hardness meter (MODELED, Schleuniger), and the average value was used as the hardness of each orally disintegrating tablet. The measurement results of hardness are shown in Table 1.

TABLE 1

| | | HPC content (%) | | | | |
|---|---|---|---|---|---|---|
| | HPC grade | 0.02 | 0.2 | 0.5 | 1.0 | 2.0 |
| Examples 1 to 5 | HPC-M | 32 | 42 | 41 | 53 | 65 |
| Examples 1 to 4 | HPC-SSL | 31 | 37 | — | 40 | 50 |
| Comparative | HPC-L | — | — | 36 | 48 | — |
| Examples 5, 6 | | | | | | |
| Comparative | HPC-H | — | — | — | 50 | — |
| Example 7 | | | | | | |

*The unit of hardness is N.

### [Disintegration Time]

The disintegration time of the orally disintegrating tablets of Examples 1 to 5 and Comparative Examples 1 to 7 was measured by a sensory test using 2 subjects, and the average value was used as the disintegration time of each orally disintegrating tablet. The measurement results of disintegration time are shown in Table 2.

TABLE 2

| | | HPC content (%) | | | | |
|---|---|---|---|---|---|---|
| | HPC grade | 0.02 | 0.2 | 0.5 | 1.0 | 2.0 |
| Examples 1 to 5 | HPC-M | 8 | 9 | 10 | 15 | 23 |
| Examples 1 to 4 | HPC-SSL | 8 | 12 | — | 23 | 42 |
| Comparative | HPC-L | — | — | 12 | 20 | — |
| Examples 5, 6 | | | | | | |
| Comparative | HPC-H | — | — | — | 21 | — |
| Examples 7 | | | | | | |

*The unit of disintegration time is seconds.

From the results of Tables 1 and 2, it was clarified that both high tablet hardness and rapid disintegration time can be achieved in Examples 1 to 5 produced by the fluid bed granulation method using HPC having a viscosity in a 2% aqueous solution at 20° C. of 150 mPa·s or more and 400 mPa·s or less. On the other hand, sufficient tablet hardness was not obtained, and a delay in disintegration time was observed in Comparative Examples 1 to 7 using other grades of HPC. Since the viscosity of the granulation liquid was high in Comparative Example 7 using HPC-H, the granulated substances became remarkably coarse, and also the production time was long.

### Example 6

Hypromellose (hereinafter also referred to as HPMC) was investigated as a binder other than HPC. As Example 6 using HPC, a pre-tableting powder was prepared by the same manufacturing method as in Example 3, and the pre-tableting powder was tableted so that the weight is 100 mg to obtain an orally disintegrating tablet having a thickness of 2.20 mm in which the content of HPC-M is 0.5 wt %.

### Comparative Example 8

As Comparative Example 8, a pre-tableting powder was prepared in the same formulation as in Comparative Example 5, and the pre-tableting powder was tableted so as to have a weight of 100 mg to obtain an orally disintegrating tablet having a thickness of 2.20 mm in which the content of HPC-L is 0.5 wt %.

### Comparative Example 9

As Comparative Example 9, an orally disintegrating tablet in which the content of HPMC is 0.5 wt % was manufactured by changing to HPMC exhibiting a viscosity similar to that of HPC-M. An orally disintegrating tablet of Comparative Example 9 having a thickness of 2.20 mm was obtained by preparing a pre-tableting powder by the same manufacturing method as in Example 3, except that 1.5 g of HPMC (METOLOSE® 65SH400, Shin-Etsu Chemical Co., Ltd., the viscosity in a 2% aqueous solution at 20° C. was 400 mPa·s) was used instead of HPC-M, and tableting the pre-tableting powder so as to have a weight of 100 mg.

### Comparative Example 10

As Comparative Example 10, an orally disintegrating tablet in which the content of HPMC is 0.5 wt % was manufactured by changing to HPMC exhibiting a viscosity similar to that of HPC-L. An orally integrating tablet of a weight of 100 mg to obtain an orally disintegrating tablet of Comparative Example 10 having a thickness of 2.20 mm was obtained by preparing a pre-tableting powder by the same manufacturing method as in Example 3, except that 1.5 g of HPMC (TC-5® R, Shin-Etsu Chemical Co, Ltd., the viscosity in a 2% aqueous solution at 20° C. was 5.2 mPa·s or more and 7 mPa·s or less) was used instead of HPC-L, and tableting the pre-tableting powder so as to have a weight of 100 mg.

For the orally disintegrating tablets of Example 6 and Comparative Examples 8 to 10, the hardness and the disintegration time were measured by the measurement method described above. The measurement results are shown in Table 3.

TABLE 3

| | | | disintegration |
|---|---|---|---|
| | Binder | hardness(N) | time(sec) |
| Example 6 | HPC-M | 24 | 9 |
| Example 8 | HPC-L | 22 | 12 |
| Comparative | HPMC: 65SH400 | 28 | 50 |
| Example 9 | | | |
| Comparative | HPMC: TC-5R | 27 | 32 |
| Example 10 | | | |

From the results in Table 3, it was clarified that the use of HPMC results in a significant delay in the disintegration time. From the disintegration time of Comparative Example 9 and Comparative Example 10, it was clarified that, in the case where HPMC is used, a delay in the disintegration time became remarkable together with an increase in the viscosity of HPMC. Therefore, it was clarified that the behavior is different depending on the relationship between the viscosity in HPC and the disintegration time of the orally disintegrating tablet and the relationship between the viscosity in HPMC and the disintegration time of the orally disintegrating tablet.

### Example 7

The effects of disintegrants added to the orally disintegrating tablet on the hardness and disintegration time were investigated. As Example 7, a pre-tableting powder was prepared by the same manufacturing method as in Example 5, and the pre-tableting powder was tableted so as to have a weight of 100 mg to obtain an orally disintegrating tablet having a thickness of 2.20 mm in which the content of HPC-M is 2.0 wt %.

### Example 8

HPC-M and L-HPC alone as the disintegrant were used to manufacture an orally disintegrating tablet of Example 8 in which the content of HPC with respect to 100 wt % of the orally disintegrating tablet is 2.0 wt %. Specifically, 6 g of HPC-M (Nippon Soda Co., Ltd.) and 1 g of sucralose (San-Ei Gen F.F.I., Inc.) were dissolved in 279 ml of purified water to prepare a granulation liquid. 249 g of D-mannitol (Mannite P, Mitsubishi Shoji Food Tech Co., Ltd.), 10 g of low-substituted hydroxypropyl cellulose (L-HPC (NBD-22), Shin-Etsu Chemical Co., Ltd.) were supplied to a fluid bed granulator (manufactured by Powrex Corporation, model: MP-01) and mixed. Fluid bed granulation was performed by spraying the granulation liquid on the mixed powder. The granulated substances were dried at 70° C. The size of the dried granulated substances was regulated by the sieve No. 22 to obtain a sized regulated powder. The sized regulated powder, 9 g of magnesium aluminometasilicate (Neusilin® UFL2, Fuji Chemical Industries Co., Ltd.), and 3 g of magnesium stearate (Taihei Chemical Industrial Co., Ltd.) were mixed to obtain a pre-tableting powder. Using a tableting machine (VELA5, manufactured by Kikusui Seisakusho Ltd.), the pre-tableting powder was tableted so as to have a weight of 100 mg to obtain an orally disintegrating tablet of Example 8 having a thickness of 2.10 mm.

### Example 9

As Example 9, a pre-tableting powder was prepared in the same formulation as in Example 8, and the pre-tableting powder was tableted so as to have a weight of 100 mg to obtain an orally disintegrating tablet having a thickness of 2.20 mm in which the content of HPC-M is 2.0 wt %.

### Example 10

HPC-M and crospovidone alone as the disintegrant were used to manufacture an orally disintegrating tablet of Example 10 in which the content of HPC with respect to 100 wt % of the orally disintegrating tablet is 2.0 wt %. Specifically, 6 g of HPC-M (Nippon Soda Co., Ltd.) and 3 g of sucralose (San-Ei Gen F.F.I. Inc.,) were dissolved in 279 ml of purified water to prepare a granulation liquid. 267 g of D-mannitol (Mannite P, Mitsubishi Shoji Food Tech Co., Ltd.) and 6 g of crospovidone (Kollidon® CL-F, BASF) were supplied to a fluid bed granulator (manufactured by Powrex Corporation, model: MP-01) and mixed. Fluid bed granulation was performed by spraying granulation liquid on the mixed powder. The granulated substances were dried at 70° C. The size of the dried granulated substances was regulated by the sieve No. 22 to obtain a size regulated powder. The size regulated powder, 6 g of crospovidone (Kollidon® CL-F, BASF), 9 g of magnesium aluminometasilicate (Neusilin® UFL2, Fuji Chemical Industries Co., Ltd.), and 3 g of magnesium stearate (Taihei Chemical Industrial Co., Ltd.) were mixed to obtain a pre-tableting powder. Using a tableting machine (VELA5, manufactured by Kikusui Seisakusho Ltd.), the pre-tableting powder was tableted so as to have a weight of 100 mg to obtain an orally disintegrating tablet of Example 10 having a thickness of 2.10 mm.

### Example 11

As Example 11, a pre-tableting powder was prepared in the same formulation as in Example 10, and the pre-tableting powder was tableted so as to have a weight of 100 mg to obtain an orally disintegrating tablet having a thickness of 2.20 mm in which the content of HPC-M is 2.0 wt %.

### Comparative Example 11

As Comparative Example 11, a pre-tableting powder was prepared by the same manufacturing method as in Comparative Example 4, and the pre-tableting powder was tableted so as to have a weight of 100 mg, thereby manufacturing an orally disintegrating tablet having a thickness of 2.20 mm in which the content of HPC-SSL is 2.0 wt %.

### Comparative Example 12

As Comparative Example 12, HPC-SSL and L-HPC alone as the disintegrant were used to manufacture an orally disintegrating tablet in which the content of HPC with respect to 100 wt % of the orally disintegrating tablet is 2.0 wt %. Using the same manufacturing method as in Example 8, except that 6 g of HPC-SSL (Nippon Soda Co., Ltd.) was used instead of HPC-M, the pre-tableting powder was tableted so as to have a weight of 100 mg to obtain an orally disintegrating tablet of Comparative Example 12 having a thickness of 2.10 mm.

### Comparative Example 13

As Comparative Example 13, a pre-tableting powder was prepared in the same formulation as in Comparative Example 12, and the pre-tableting powder was tableted so as to have a weight of 100 mg to obtain an orally disintegrating tablet having a thickness of 2.20 mm in which the content of HPC-SSL is 2.0 wt %.

### Comparative Example 14

As Comparative Example 14, HPC-SSL and crospovidone alone as the disintegrant were used to manufacture the orally disintegrating tablet in which the content of HPC with respect to 100 wt % of the orally disintegrating tablet is 2.0 wt %. Using the same manufacturing method as in Example 10, except that 6 g of HPC-SSL (Nippon Soda Co., Ltd.) was used instead of HPC-M, the pre-tableting powder was tableted so as to have a weight of 100 mg to obtain an orally disintegrating tablet of Comparative Example 14 having a thickness of 2.10.

### Comparative Example 15

As Comparative Example 15, a pre-tableting powder was prepared in the same formulation as in Comparative Example 14, and the pre-tableting powder was tableted so as to have a weight of 100 mg to obtain an orally disintegrating tablet having a thickness of 2.20 mm in which the content of HPC-SSL is 2.0 wt %.

For the orally disintegrating tablets of Examples 7 to 11 and Comparative Examples 11 to 15, the hardness and the disintegration time were measured by the measurement method described above. The measurement results of Examples 8 and 10 and Comparative Examples 12 and 14 which are orally disintegrating tablets having a thickness of 2.10 mm are shown in Table 4 together with the results of Example 5 and Comparative Example 4. The measurement results of Examples 7, 9, 11 and Comparative Examples 11, 13, and 15 which are orally disintegrating tablets having a thickness of 2.20 mm are shown in Table 5.

TABLE 4

| | | | Hardness | Disintegration |
|---|---|---|---|---|
| | HPC grade | disintegrant | (N) | time (sec) |
| Example 5 | HPC-M | L-HPC, | 65 | 23 |
| | | carmellose, | | |
| | | crospovidone | | |
| Comparative | HPC-SSL | L-HPC, | 50 | 42 |
| Example 4 | | carmellose, | | |
| | | crospovidone | | |
| Example 8 | HPC-M | L-HPC | 52 | 40 |
| Comparative | HPC-SSL | L-HPC | 49 | 61 |
| Example 12 | | | | |
| Example 10 | HPC-M | crospovidone | 60 | 22 |
| Comparative | HPC-SSL | crospovidone | 55 | 48 |
| Example 14 | | | | |

TABLE 5

| | | | Hardness | Disintegration |
|---|---|---|---|---|
| | HPC grade | disintegrant | (N) | time (sec) |
| Example 7 | HPC-M | L-HPC, | 41 | 16 |
| | | carmellose, | | |
| | | crospovidone | | |
| Comparative | HPC-SSL | L-HPC, | 31 | 28 |
| Example 11 | | carmellose, | | |
| | | crospovidone | | |
| Example 9 | HPC-M | L-HPC | 37 | 32 |
| Comparative | HPC-SSL | L-HPC | 32 | 36 |
| Example 13 | | | | |
| Example 11 | HPC-M | crospovidone | 41 | 15 |
| Comparative | HPC-SSL | crospovidone | 35 | 28 |
| Example 15 | | | | |

From the results of Tables 4 and 5, it was clarified that, even if the disintegrant is changed, higher hardness and rapid disintegration can be obtained in the Examples using HPC-M compared with the Comparative examples using HPC-SSL. In addition, it was clarified that, even if the thickness of the tablet is changed, higher hardness and rapid disintegration can be obtained in the Examples using HPC-M compared with the Comparative examples using HPC-SSL. Therefore, it was clarified that the binder added to the granulation liquid used for the fluid bed granulation has a greater effect than the disintegrant in order to obtain high hardness and rapid disintegration in the orally disintegrating tablet.

### Comparative Example 16

The effects of different manufacturing methods, especially different granulation methods, on hardness and disintegration time were investigated. As Comparative Example 16, HPC-M was added as a powder at the time of fluid bed granulation to manufacture an orally disintegrating tablet in which the content of HPC with respect to 100 wt % of the orally disintegrating tablet is 2.0 wt %. Specifically, 3 g of sucralose (San-Ei Gen F.F.I., Inc.) was dissolved in 276 ml of purified water to prepare a granulation liquid. 6 g of HPC-M (Nippon Soda Co., Ltd.), 237 g of D-mannitol (Mannite P, Mitsubishi Shoji Food Tech Co., Ltd.), 18 g of low-substituted hydroxypropyl cellulose (L-HPC (NBD-22), Shin-Etsu Chemical Co., Ltd.) were supplied to a fluid bed granulator (manufactured by Powrex Corporation, model: MP-01) and mixed. Fluidized bed granulation was performed by spraying granulation liquid on the mixed powder. The granulated substances were dried at 70° C. The size of the dried granulated substances was regulated by the sieve No. 22 to obtain a size regulated powder. The size regulated powder, 6 g of crospovidone (Kollidon® CL-F, BASF), 9 g of magnesium aluminometasilicate (Neusilin® UFL2, Fuji Chemical Industries Co., Ltd.), and 3 g of magnesium stearate (Taihei Chemical Industrial Co., Ltd.) were mixed to obtain a pre-tableting powder. Using a tableting machine (VELA5, manufactured by Kikusui Seisakusho Ltd.), the pre-tableting powder was tableted so as to have a weight of 100 mg to obtain an orally disintegrating tablet of Comparative Example 16 having a thickness of 2.10 mm.

### Comparative Example 17

As Comparative Example 17, HPC-SSL was used to manufacture an orally disintegrating tablet in which the content of HPC with respect to 100 wt % of the orally disintegrating tablet is 2.0 wt % by the same manufacturing method as in Comparative Example 16. Using the same manufacturing method as in Comparative Example 16, except that 6 g of HPC-SSL (Nippon Soda Co., Ltd.) was used instead of HPC-M, the pre-tableting powder was tableted so as to have a weight of 100 mg to obtain an orally disintegrating tablet of Comparative Example 17 having a thickness of 2.10 mm.

### Comparative Example 18

As Comparative Example 18, HPC-M was used to manufacture an orally disintegrating tablet in which the content of HPC with respect to 100 wt % the orally disintegrating tablet is 2.0 wt % by a direct compressing method. Specifically, 79 g of D-mannitol (PEARLITOL 200SD, ROQUETTE FRERES), 6 g of low-substituted hydroxypropyl cellulose (L-HPC (NBD-22), Shin-Etsu Chemical Co., Ltd., 2 g of HPC-M (Nippon Soda Co., Ltd.), 1 g of sucralose (San-Ei Gen F.F.I., Inc.), 6 g of carmellose (NS-300®), 2 g of crospovidone (Kollidon® CL-F, BASF), and 3 g of magnesium aluminometasilicate (Neusilin® UFL2, Fuji Chemical Industries Co., Ltd.) were mixed in a vinyl bag and sieved by the sieve No. 30. 1 g of magnesium stearate (Taihei Chemical Industrial Co., Ltd.) was mixed into the mixed powder to obtain a pre-tableting powder. Using a tableting machine (VELA5, manufactured by Kikusui Seisakusho Ltd.), the pre-tableting powder was tableted so as to have a weight of 100 mg to obtain an orally disintegrating tablet of Comparative Example 18 having a thickness of 2.10 mm.

### Comparative Example 19

As Comparative Example 19, HPC-SSL was used to manufacture an orally disintegrating tablet in which the content of HPC with respect to 100 wt % of the orally disintegrating tablet is 2.0 wt % by the same manufacturing method as in Comparative Example 18. Using the same manufacturing method as in Comparative Example 18, except that 2 g of HPC-SSL (Nippon Soda Co., Ltd.) was used instead of HPC-M, the pre-tableting powder was tableted so as to have a weight of 100 mg to obtain an orally disintegrating tablet of Comparative Example 19 having a thickness of 2.10 mm.

### Comparative Example 20

As Comparative Example 20, HPC-M was used to manufacture an orally disintegrating tablet in which the content of HPC with respect to 100 wt % of the orally disintegrating tablet is 2.0 wt % by a kneading method. Specifically, 237 g of D-mannitol (Mannite P, Mitsubishi Shoji Food Tech Co., Ltd.), 18 g of low-substituted hydroxypropyl cellulose (L-HPC (NBD-22), Shin-Etsu Chemical Co., Ltd.), 3 g of sucralose (San-Ei Gen F.F.I., Inc.), and 18 g of HPC-M (Nippon Soda Co., Ltd.) were mixed in a vinyl bag and sieved by the sieve No. 30. 41.8 ml of purified water was added to the sieved mixed powder and kneaded using a high-speed mixer (LFS-GS-2J, manufactured by EARTHTECHNICA CO., LTD.). The obtained kneaded product was heated in a mini jet oven (manufactured by fujimak corporation) for 3 hours at 70° C., and dried. The size of the dried mixture was regulated with a power mill φ1 mm (manufactured by DALTON CORPORATION). The size regulated powder, 6 g of crospovidone (Kollidon® CL-F, BASF), 9 g of magnesium aluminometasilicate (Neusilin® UFL2, Fuji Chemical Industries Co., Ltd.), and 3 g of magnesium stearate (Taihei Chemical Industrial Co., Ltd.) were mixed to obtain a pre-tableting powder. Using a tableting machine (VELA5, manufactured by Kikusui Seisakusho Ltd.), the pre-tableting powder was tableted so as to have a weight of 100 mg to obtain an orally disintegrating tablet of Comparative Example 20 having a thickness of 2.10 mm.

### Comparative Example 21

As Comparative Example 21, HPC-SSL was used to manufacture an orally disintegrating tablet in which the content of HPC with respect to 100 wt % of the orally disintegrating tablet is 2.0 wt % by the same manufacturing method as in Comparative Example 20. Using the same manufacturing method as in Comparative Example 20, except that 6 g of HPC-SSL (Nippon Soda Co., Ltd.) was used instead of HPC-M and purified water was changed to 57.7 ml, the pre-tableting powder was tableted so as to a weight of 100 mg to obtain an orally disintegrating tablet of Comparative Example 21 having a thickness of 2.10 mm.

For the orally disintegrating tablets of Comparative Examples 16 to 21, the hardness and the disintegration time were measured by the measurement method described above. The measurement results of Comparative Examples 16 to 21 are shown in Table 6 together with the results of Example 5 and Comparative Example 4.

TABLE 6

| | | Granulation | Hardness | Disintegration |
|---|---|---|---|---|
| | HPC grade | method | (N) | time (sec) |
| Example 5 | HPC-M | Fluid bed | 65 | 23 |
| | | granulation | | |
| | | (Liquid | | |
| | | addition) | | |
| Comparative | HPC-SSL | Fluid bed | 50 | 42 |
| Example 4 | | granulation | | |
| | | (Liquid | | |
| | | addition) | | |
| Comparative | HPC-M | Fluid bed | 33 | 10 |
| Example 16 | | granulation | | |
| | | (Powder | | |
| | | addition) | | |
| Comparative | HPC-SSL | Fluid bed | 37 | 16 |
| Example 17 | | granulation | | |
| | | (Powder | | |
| | | addition) | | |
| Example 18 | HPC-M | direct | 88 | 37 |
| | | compressing | | |
| Comparative | HPC-SSL | direct | 94 | 46 |
| Example 19 | | compressing | | |
| Example 20 | HPC-M | kneading | 27 | 11 |
| Comparative | HPC-SSL | kneading | 40 | 38 |
| Example 21 | | | | |

From the results of Table 6, it was clarified that the orally disintegrating tablet using HPC-M has a shorter orally disintegrating time than the orally disintegrating tablet using HPC-SSL regardless of the manufacturing method. On the other hand, for the hardness of the orally disintegrating tablets, only the orally disintegrating tablets, which were fluid bed sized by adding a liquid of HPC-M, exhibited higher hardness than the orally disintegrating tablets using HPC-SSL. Therefore, it was clarified that higher hardness and rapid disintegration than an orally disintegrating tablet using HPC-SSL can be obtained by adding a liquid of HPC-M and performing fluid bed granulation.

### [Bulk Density of Size Regulated Powder]

The size regulated powders before tableting of Example 5, Comparative Example 4, and Comparative Examples 16 to 21 were filled into a container having a volume of 100 ml without tapping, and the mass thereof were measured to calculate the bulk density. The measured bulk densities are shown in Table 7.

TABLE 7

| | | | Bulk |
|---|---|---|---|
| | | Granulation | density |
| | HPC grade | method | (g/mL) |
| Example 5 | HPC-M | Fluid bed | 0.32 |
| | | granulation | |
| | | (Liquid | |
| | | addition) | |
| Comparative | HPC-SSL | Fluid bed | 0.31 |
| Example 4 | | granulation | |
| | | (Liquid | |
| | | addition) | |
| Comparative | HPC-M | Fluid bed | 0.47 |
| Example 16 | | granulation | |
| | | (Powder | |
| | | addition) | |
| Comparative | HPC-SSL | Fluid bed | 0.46 |
| Example 17 | | granulation | |
| | | (Powder | |
| | | addition) | |
| Comparative | HPC-M | direct | 0.52 |
| Example 18 | | compressing | |
| Comparative | HPC-SSL | direct | 0.52 |
| Example 19 | | compressing | |
| Comparative | HPC-M | kneading | 0.55 |
| Example 20 | | | |
| Comparative | HPC-SSL | kneading | 0.48 |
| Example 21 | | | |

From the bulk densities shown in Table 7, it was clarified that the bulk density of the size regulated powder of Example 5 obtained by fluid bed granulating using HPC-M in the granulation liquid was 0.40 g/cm³ or less, which was smaller than that of the size regulated powder produced by other manufacturing methods. It is presumed that the size regulated powder obtained by granulating using a granulation liquid including HPC having a viscosity in a 2% aqueous solution at 20° C. of 150 mPa·s or more and 400 mPa·s or less has a bulk density of 0.40 g/cm³ or less, so that it is possible to have adequate hardness for preventing breakage of the tablet during transport or handling by a medical practitioner or a patient while rapid disintegration is maintained.

According to an embodiment of the present invention, a method for manufacturing an orally disintegrating tablet having adequate hardness for preventing breakage of the tablet during transport or handling by a medical practitioner or a patient is provided while rapid disintegration is maintained.

1. A method for manufacturing an orally disintegrating tablet comprising:
performing fluid bed granulation while spraying or dropping a liquid including hydroxypropyl cellulose having a viscosity in a 2% aqueous solution at 20° C. of 150 mPa·s or more and 400 mPa·s or less on additives.

2. The method for manufacturing the orally disintegrating tablet according to claim 1, wherein
the liquid including the hydroxypropyl cellulose is sprayed or dropped on the additives so that a content of the hydroxypropyl cellulose with respect to 100 wt % of the orally disintegrating tablet is 0.2 wt % or more and 5 wt % or less.

3. The method for manufacturing the orally disintegrating tablet according to claim 1, wherein
the liquid including the hydroxypropyl cellulose is sprayed or dropped on the additives so that the content of the hydroxypropyl cellulose with respect to 100 wt % of the orally disintegrating tablet is 0.2 wt % or more and 2 wt % or less.

4. The method for manufacturing the orally disintegrating tablet according to claim 1, wherein
the liquid including the hydroxypropyl cellulose is prepared by dissolving hydroxypropyl cellulose and a sweetener; and
the additives are prepared by mixing an excipient and a disintegrant.

5. The method for manufacturing the orally disintegrating tablet according to one of claim 1, wherein
a pharmaceutically active ingredient is dissolved or dispersed in the liquid including the hydroxypropyl cellulose, or
the pharmaceutically active ingredient is mixed with the additives.

6. An orally disintegrating tablet, comprising:
a particle including hydroxypropyl cellulose having a viscosity in a 2% aqueous solution at 20° C. of 150 mPa·s or more and 400 mPa·s or less,
wherein
a bulk density of the particle is 0.40 g/cm³ or less.

7. The orally disintegrating tablet according to claim 6, wherein
a content of the hydroxypropyl cellulose with respect to 100 wt % of the orally disintegrating tablet is 0.2 wt % or more and 5 wt % or less.

8. The orally disintegrating tablet according to claim 6, wherein
a content of the hydroxypropyl cellulose with respect to 100 wt % of the orally disintegrating tablet is 0.2 wt % or more and 2 wt % or less.
